(19) United States
(12) Patent Application Publication
(10) Pub. No.: US 2018/0221995 A1
(43) Pub. Date: Aug. 9, 2018

(54) Au-BASED SOLDERING BALL, CERAMIC ELECTRONIC COMPONENT SEALED OR BONDED THEREWITH, AND METHOD FOR EVALUATING BONDING RELIABILITY OF SAID Au-BASED SOLDERING BALL

(71) Applicant: SUMITOMO METAL MINING CO., LTD., Tokyo (JP)

(72) Inventor: Eiji MURASE, Ome-shi (JP)

(73) Assignee: SUMITOMO METAL MINING CO., LTD., Tokyo (JP)

(21) Appl. No.: 15/577,457

(22) PCT Filed: Mar. 25, 2016
(86) PCT No.: PCT/JP2016/059626

§ 371 Date: Apr. 12, 2018

(30) Foreign Application Priority Data
May 29, 2015 (JP) 2015-110842

(51) Int. Cl. B23K 35/30; B23K 35/02; C22C 5/02; G01N 3/08; G01N 33/20; H01L 23/10

(52) U.S. Cl. CPC B23K 35/3013; B23K 35/0222; H01L 23/10; G01N 3/08; G01N 33/20; C22C 5/02

(57) ABSTRACT

Provided is an Au-based solder alloy that has high bonding reliability even when its Au content is lower than that of a conventional Au-based solder alloy.

The Au-based solder alloy is used for sealing or bonding a ceramic electronic component and made of an Au—Sn—Ag alloy containing 21.1 to 43.0% by mass of Sn, 0.1 to 15% by mass of Ag, and a balance of Au and inevitable impurities, an Au—Ge—Sn alloy containing 9.5 to 15% by mass of Ge, 2 to 10% by mass of Sn, and a balance of Au and inevitable impurities, or an Au—Ag—Ge alloy containing 5 to 18% by mass of Ag, 7 to 20% by mass of Ge, and a balance of Au and inevitable impurities. When the Au-based soldering ball is crushed in one direction, a maximum stress before cracking occurs is 2.0×10² N/mm² or more, and a square root of strain before the cracking occurs is 0.40 or more.

### TECHNICAL FIELD

The present invention relates to an Au-based soldering ball used for sealing or bonding a ceramic electronic component which needs to have airtightness such as a quartz crystal unit, a ceramic electronic component sealed or bonded using the soldering ball, and a method for evaluating the bonding reliability of the Au-based soldering ball.

### BACKGROUND ART

Electronic devices, such as communication devices and office automation equipment, use electronic components such as quartz crystal units, crystal oscillators, and SAW filters. These electronic components are required to have high reliability and high airtightness because of their structure. For example, when an electronic component is bonded or sealed using a solder material, the solder material is required to have sufficient reliability for bonding or sealing (hereinafter, also referred to as solderability) so that oxygen and moisture do not enter the inside of the electronic component or the inside of the solder material used for bonding or sealing. In order to achieve such sufficient solderability, a soldering ball used for bonding or sealing an electronic component is made of an expensive Au-based alloy such that an oxide layer to be formed on its surface should be as thin as possible which thus improves wettability.

For example, Patent Literature 1 discloses a technique in which the Ge content of the surface layer of an Au—Ge alloy soldering ball is reduced to prevent the soldering ball from decreasing in solderability which is caused by surface oxidation. Further, Patent Literature 2 discloses an Au—Ge alloy ball for soldering which is excellent in flowability and wettability so that molten solder uniformly wets and spreads. The Au—Ge alloy has a relatively high melting point, and therefore there is a case where an element inside a package is thermally damaged during sealing. Therefore, for example, Patent Literature 3 discloses a technique in which an Au—Ga—In alloy having a lower melting point than the Au—Ge alloy is used as a solder material. It is to be noted that the reliability of an Au solder alloy is evaluated by a heat cycle test in which a device bonded using the Au-based solder alloy is repeatedly heated and cooled in order to alternately apply the stresses of expansion and contraction to the device or by a long-term storage test in which the device is exposed to a high- or low-temperature environment in order to apply the stress of expansion or contraction to the device.

### CITATION LIST

### Patent Literature

Patent Literature 1: JP2008-030093A

Patent Literature 2: JP2010-214396A

Patent Literature 3: WO2010/010833

### SUMMARY OF INVENTION

### Technical Problem

As described above, Au-based solder alloys are conventionally used for sealing or bonding electronic components required to have high reliability and airtightness, such as quartz crystal units, crystal oscillators, SAW filters, and gyroscope sensors. With recent widespread use of communication devices such as mobile phones, such electronic components have come to be widely used not only in relatively expensive communication devices but also in common communication devices for civil use. Therefore, solder alloys used for electronic components are strongly required to reduce their cost by, for example, reducing the content of expensive Au.

However, in the case of, for example, a solder alloy for hermetic sealing, simply reducing its Au content may lead to the loss of airtightness, because when the solder alloy is solidified, microcracking may occur due to the application of stress caused by a difference in thermal expansion between the solder alloy and a package material. Such a difference in thermal expansion is remarkable especially when the package material is ceramic. Therefore, solder materials that are less likely to cause cracking have been developed, and the mechanical properties of such solder materials are evaluated by, for example, a tensile test such as one specified in JIS Z 3198-2 (2003).

With recent downsizing and upgrading of electronic components, the sizes of materials used for sealing or bonding have also become smaller and smaller. However, the above-described tensile test is performed using a relatively large test specimen, and therefore the properties of such a small-sized solder material to be practically used for electronic components are sometimes not correctly evaluated. Further, when the size of a test specimen is reduced in line with the size of a solder alloy to be practically used, residual stress induced by a fabrication process of the test specimen has a great effect on test results. Therefore, also in this case, the properties of the solder alloy to be practically used, such as the ease of occurrence of cracking, are sometimes not correctly evaluated.

In light of the above circumstances, it is an object of the present invention to provide a method for evaluating an Au-based solder alloy having a lower Au content in comparison with a conventional Au-based solder alloy as to whether cracking is likely to occur or not in a simple and reproducible manner, an Au-based solder alloy having high bonding reliability that is identified by the evaluation method, and an electronic component bonded or sealed using the Au-based solder alloy.

### Solution to Problem

In order to achieve the above object, the present invention is directed to an Au-based soldering ball used for sealing or bonding a ceramic electronic component, wherein when the soldering ball is crushed in one direction, a maximum stress before cracking occurs is 2.0×10² N/mm² or more, and a square root of strain before the cracking occurs is 0.40 or more.

### Advantageous Effects of Invention

According to the present invention, it is possible to provide an Au-based solder alloy that is less likely to cause microcracking. The use of such an Au-based solder alloy for sealing or bonding an electronic component typified by a quartz crystal unit, a crystal oscillator, a SAW filter, or a gyroscope sensor makes it possible to provide the electronic component having high reliability due to excellent hermetical sealing and excellent bonding. Further, it is possible to easily determine whether an Au-based solder alloy composition has high reliability or not, which makes it possible to eliminate the need to perform a time-consuming reliability verification test and thus promote the development of Au-based solder alloys having a reduced Au content.

### BRIEF DESCRIPTION OF DRAWING

FIG. 1 A schematic front view for explaining a test method for evaluating the amount of displacement of a soldering ball.

### DESCRIPTION OF EMBODIMENTS

Hereinbelow, an Au-based soldering ball according to the present invention will be described in detail. The present inventor has examined various electronic components sealed or bonded using Au-based solder alloys to determine the occurrence of microcracking in the Au-based solder alloys which affects the reliability of the electronic components, and as a result has found that microcracking occurs near and along the bonding interface between the Au-based solder alloy and the electronic component. It is considered that the occurrence of microcracking is caused by strain of a soldering alloy that exceeds the fracture strength of the solder alloy, in which the strain is caused by contraction of the solder alloy during solidification after an electronic component is sealed with the solder alloy in molten state.

The present inventor has intensively studied on the basis of the examination, and as a result has found that there is a very strong positive correlation between strain caused by tensile stress due to the contraction of an Au-based solder alloy and strain caused when a ball-shaped solder alloy is compressed in one direction. More specifically, the present inventor has found that when Au-based soldering balls different in composition from one another are each placed on a flat floor surface and then gradually pressed from directly above by a pressing member having a pressing surface parallel with the floor surface, each of the soldering balls is gradually crushed and spread into a disk shape along the floor surface, and then when it is crushed to some extent, cracking occurs in the outer periphery of the disk along the circumferential direction of the disk.

Further, the present inventor has examined solder joints using the same Au-based soldering balls as used in the above test, respectively, and as a result has found that the ease of occurrence of cracking can be easily estimated based on a parameter of stress and strain at the time of fracture by cracking when the soldering ball is crushed. Specifically, a soldering ball can withstand thermal contraction caused by general solder bonding or sealing on condition that its maximum stress before the occurrence of cracking is 2.0×10² N/mm² or more and its square root of strain before the occurrence of the cracking is 0.40 or more when the soldering ball is crushed in one direction. Therefore, Evaluation of an previously-sampled Au-based solder alloy according to the above criteria makes it possible to provide the Au-based soldering ball that is less likely to cause microcracking even though the Au content of an Au-based solder alloy is reduced as compared to that of a conventional Au-based solder alloy.

The above-described parameterization can be achieved by the following reason. Supposing that a column-shaped solder alloy is crushed to a height of 1/a by pressing it in its center axis direction, the volume of the solder alloy itself is not changed, and therefore the area of the cross section perpendicular to the center axis is increased a-fold, and the diameter of the cross section is √a-fold that before crush. It can be considered that the same applies to a case where a soldering ball is crushed by pressing it in one direction. Further, it can be considered that the ease of occurrence of cracking of a soldering ball when it is crushed in one direction is influenced in proportion to √a, because as described above, when a soldering ball is crushed into a disk shape, cracking occurs in the outer periphery of the disk.

Defining that ‘dD’ is an amount of displacement in a pressing direction of a solder ball having a diameter of ‘D’ when it is crushed by pressing in one direction, the above-described ‘a’ can be expressed as a=D/(D−dD). Further, dD/D can be regarded as strain ‘ε’, and therefore the above-described ‘a’ can be expressed as a=1/(1−ε). In light of the above points, it is considered that the ease of occurrence of cracking of a soldering ball when it is crushed by pressing it in one direction can be parameterized by using the square root of strain ‘ε’. Therefore, the present inventor has performed experiments using various Au-based soldering balls, and as a result has found that, as described above, Au-based solder alloys that can sufficiently withstand thermal contraction during solder bonding or sealing can be identified by evaluation based on the maximum stress and the square root of strain before the cracking. Therefore, even when the Au content of a solder alloy is reduced, an Au-based soldering ball that is less likely to cause cracking and has high bonding reliability can be provided by performing a test in advance using the above-described parameter.

A soldering ball having maximum stress before the occurrence of cracking of less than 2.0×10² N/mm² is not preferable. This is because when such a soldering ball is used for solder bonding or sealing, there may be a case that the soldering ball is easily damaged by an external factor other than thermal contraction, such as physical impact. Further, a soldering ball having square root of strain of less than 0.40 before the occurrence of cracking is not preferable. This is because there may be a case that the soldering ball is immediately damaged due to thermal contraction during solder bonding or sealing, or the soldering ball is damaged due to internal strain when heating and cooling are alternately repeated or when thermal stress is applied for a long time even in a constant temperature environment, and therefore reliability is likely to be impaired.

The above-described parameterization for evaluation of the ease of occurrence of cracking is based on the results of the experiment performed using Au-based soldering balls of various compositions, and therefore can basically be applied only to Au-based soldering balls. Hereinbelow, an Au—Sn—Ag alloy, an Au—Ge—Sn alloy, and an Au—Ag—Ge alloy will specifically be described to which the above-described method for evaluating the ease of occurrence of cracking can be applied.

### <Au—Sn—Ag Alloy>

A first Au-based solder alloy to which the above-described evaluation method can be applied is an Au—Sn—Ag alloy. This Au—Sn—Ag alloy has an Sn content of 21.1% by mass or more but 43.0% by mass or less. If the Sn content is less than 21.1% by mass, crystal grains are too large, which is not preferred because the requirement of strain ε described above cannot be satisfied. On the other hand, if the Sn content is more than 43.0% by mass, wettability is deteriorated, which is not preferred because there is a case where cracking occurs at the interface between an electronic component and solder.

This Au—Sn—Ag alloy has an Ag content of 0.1% by mass or more but 15% by mass or less. If the Ag content is less than 0.1% by mass, crystal grains are too large, which is not preferred because the requirement of strain ε described above cannot be satisfied. Also, even if the Ag content is more than 15% by mass, crystal grains are too large, which is not preferred because the requirement of strain ε cannot be satisfied.

### <Au—Ge—Sn Alloy>

A second Au-based solder alloy to which the above-described evaluation method can be applied is an Au—Ge—Sn alloy. This Au—Ge—Sn alloy has a Ge content of 9.5% by mass or more but 15% by mass or less. If the Ge content is less than 9.5% by mass, the difference between a liquidus temperature and a solidus temperature is too large, which is not preferred because a phenomenon occurs in which the components of the alloy are separated from one another. Also, even if the Ge content is more than 15% by mass, the difference between a liquidus temperature and a solidus temperature is too large, which is not preferred because a phenomenon occurs in which the components of the alloy are separated from one another.

This Au—Ge—Sn alloy has an Sn content of 2% by mass or more but 10% by mass or less. If the Sn content is less than 2% by mass, crystal grains are too large, which is not preferred because the requirement of strain ε described above cannot be satisfied. On the other hand, if the Sn content is more than 10% by mass, wettability is deteriorated, which is not preferred because there is a case where cracking occurs at the interface between an electronic component and solder.

### <Au—Ag—Ge Alloy>

A third Au-based solder alloy to which the above-described evaluation method can be applied is an Au—Ag—Ge alloy. This Au—Ag—Ge alloy has an Ag content of 5% by mass or more but 18% by mass or less. If the Ag content is less than 5% by mass, crystal grains are too large, which is not preferred because the requirement of strain ε described above cannot be satisfied. Also, even if the Ag content is more than 18% by mass, crystal grains are too large, which is not preferred because the requirement of strain ε described above cannot be satisfied.

This Au—Ag—Ge alloy has a Ge content of 7% by mass or more but 20% by mass or less. If the Ge content is less than 7% by mass, the difference between a liquidus temperature and a solidus temperature is too large, which is not preferred because a phenomenon occurs in which the components of the alloy are separated from one another. Also, even if the Ge content is more than 20% by mass, the difference between a liquidus temperature and a solidus temperature is too large, which is not preferred because a phenomenon occurs in which the components of the alloy are separate from one another.

### EXAMPLES

Hereinbelow, the present invention will be described in more detail with reference to specific examples, but is not limited by these examples at all. First, Au, Sn, Ag, and Ge each having a purity of 99.9% by mass or more were prepared as raw materials. Large flaky or bulky raw materials were cut or ground into small pieces of 3 mm or less so that the composition of a resulting molten alloy was made uniform without variations from sampling point to sampling point. Then, a predetermined amount of each of these raw materials was weighed and placed in a graphite crucible for high-frequency melting furnace.

The crucible containing the raw materials was placed in a high-frequency melting furnace, and nitrogen was flowed at a flow rate of 0.7 L/min or more per kilogram of the raw materials in order to prevent oxidation. In this state, the melting furnace was turned on to heat and melt the raw materials. When starting to melt, the metals were well stirred with a stirring stick so that the composition of a molten metal was made uniform without local variations. After it was confirmed that the metals were fully melted, the high-frequency melting furnace was turned off, the crucible was immediately taken out of the high-frequency melting furnace, and the molten metal in the crucible was poured into a mold for solder master alloy. As the mold, a column-shaped mold having a diameter of 24 mm was used. In this way, a solder alloy ingot was prepared as Sample 1.

Further, solder alloy ingots different in composition were prepared as Samples 2 to 28 in the same manner as described above with reference to Sample 1 except that the mixing ratio among the raw materials weighed and placed in the graphite crucible was variously changed. It is to be noted that Sample 26 is a conventionally-used Ge alloy having an Au content of 12.5% by weight. The composition of each of the thus obtained solder alloys of Samples 1 to 28 was analyzed using an ICP emission spectrophotometer (SHIMADZU S-8100). The analytical results are shown in the following Table 1.

TABLE 1

Composition (% by mass)

| Samples | Au | Sn | Ge | Ag |
|---|---|---|---|---|
| 1 | 60 | 30 | — | 10 |
| 2 | 77.9 | 21.1 | — | 1 |
| 3 | 42 | 43 | — | 15 |
| *4 | 70 | 20 | — | 10 |
| *5 | 46 | 44 | — | 10 |
| *6 | 54 | 30 | — | 16 |
| *7 | 40 | 44 | — | 16 |
| 8 | 83 | 5 | 12 | — |
| 9 | 88 | 2 | 10 | — |
| 10 | 75 | 10 | 15 | — |
| *11 | 87 | 1 | 15 | — |
| *12 | 77 | 11 | 12 | — |
| *13 | 86 | 5 | 9 | — |
| *14 | 79 | 5 | 16 | — |
| *15 | 90 | 1 | 9 | — |
| *16 | 73 | 11 | 16 | — |
| 17 | 77 | — | 10 | 13 |
| 18 | 88 | — | 7 | 5 |
| 19 | 62 | — | 20 | 18 |
| *20 | 86 | — | 6 | 8 |
| *21 | 71 | — | 21 | 8 |
| *22 | 82 | — | 14 | 4 |
| *23 | 75 | — | 14 | 19 |
| *24 | 90 | — | 6 | 4 |
| *25 | 68 | — | 21 | 20 |
| *26 | 87.5 | — | 12.5 | — |
| 27 | 83 | 5 | 12 | — |
| 28 | 83 | 5 | 12 | — |

(Note)
The samples marked with * in the table are comparative examples.

### Example 1

### <Production of Ball-Shaped Solder Alloys>

Out of the solder alloy ingots of Samples 1 to 28, each of the solder alloy ingots of Samples 1 to 26 was charged in a nozzle of a liquid-phase atomization apparatus, and the nozzle was set at the upper part of a quartz tube (inside a high-frequency melting coil) containing oil heated to 330° C. Then, the ingot in the nozzle was heated to 500° C. by high frequency and maintained at this temperature for 5 minutes, and was then atomized by pressurizing the nozzle with an inert gas to produce balls of the solder alloy. It is to be noted that the inside diameter of the nozzle tip was previously adjusted so that the balls formed by this atomization process had a diameter of 0.25 mm.

The obtained soldering balls of each of the samples were washed with ethanol three times and then dried in vacuum at 40° C. for 3 hours in a vacuum drier. The obtained soldering balls of each of the samples were measured with a measuring microscope STM-5 manufactured by Olympus Corporation to select soldering balls having an outer diameter of 0.25 mm.

### <Ball Ductility Evaluation Test>

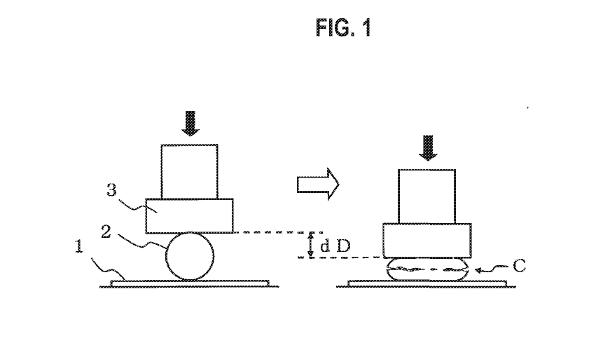

Next, the soldering ball of each of the samples was crushed into a disk shape by applying a load with an indenter with the use of a micro strain tester MST-1 manufactured by SHIMADZU CORPORATION to measure the maximum stress and the amount of displacement before cracking occurred. More specifically, as shown in FIG. 1, a soldering ball 2 of each of the samples was placed on a silicon wafer 1, and the position of the soldering ball 2 was finely adjusted so that the soldering ball 2 abutted against the center of an indenter 3 of the tester. Then, the indenter 3 was brought into contact with the soldering ball 2 to such an extent that a heavy load was not applied to the soldering ball 2. Then, the soldering ball 2 was crushed into a disk shape by pressing it with the indenter 3 in one direction indicated by a black arrow at a pressing rate of 1 mm/min to measure the maximum load and the amount of displacement dD before cracking C occurred in the outer periphery of the disk.

### <Bonding Reliability Evaluation Test>

In order to evaluate the reliability of solder bonding, the soldering ball of each of the samples was soldered to a Ni-plated Cu substrate to prepare a joined body, and the joined body was subjected to a heat cycle test. More specifically, an Ni-plated (film thickness: 3.0 μm) Cu substrate (plate thickness: 0.3 mm) was heated in a nitrogen atmosphere for 25 seconds, and then the soldering ball of each of the samples was placed on the Cu substrate and heated for 25 seconds. After the completion of the heating for 25 seconds, the Cu substrate joined with the soldering ball was sufficiently cooled in a nitrogen atmosphere and then exposed to the atmosphere.

The thus obtained joined body was subjected to a predetermined number of repetitions of a cycle of cooling at −40° C. and heating at 150° C. Then, the Cu substrate joined with the solder alloy was embedded in a resin, and the cross section of the resin was polished to observe a bonding interface by SEM (S-4800 manufactured by Hitachi, Ltd.). The bonding interface was evaluated according to the following criteria: “Failed”: separation at the bonding interface or cracking of solder was observe; and “Passed”: the bonding interface remained in its initial state without such defects. The result of the heat cycle test of the joined body is shown in the following Table 2 together with a maximum stress determined by dividing the maximum load by the area of the cross section passing through the center of the soldering ball before crush, a strain ε determined by dividing the amount of displacement dD by the diameter D of the soldering ball before crush, and a square root of the strain ε.

TABLE 2

| | Ball | Cross-sectional | Maximum | Maximum | Amount of | | | |
|---|---|---|---|---|---|---|---|---|
| | diameter | area | load | stress | displacement | Strain ε | √ε | |
| Samples | [mm] | [mm²] | [mN] | [N/mm²] | [mm] | [—] | [—] | Evaluation |
| 1 | 0.25 | 0.0491 | 20,348 | 415 | 0.0665 | 0.27 | 0.52 | Passed |
| 2 | 0.25 | 0.0491 | 15,740 | 321 | 0.0587 | 0.23 | 0.48 | Passed |
| 3 | 0.25 | 0.0491 | 12,472 | 254 | 0.0426 | 0.17 | 0.41 | Passed |
| *4 | 0.25 | 0.0491 | 10,332 | 210 | 0.0355 | 0.14 | 0.38 | Failed |
| *5 | 0.25 | 0.0491 | 9,612 | 196 | 0.0398 | 0.16 | 0.40 | Failed |
| *6 | 0.25 | 0.0491 | 12,965 | 264 | 0.0389 | 0.16 | 0.39 | Failed |
| *7 | 0.25 | 0.0491 | 8,863 | 181 | 0.0218 | 0.09 | 0.30 | Failed |
| 8 | 0.25 | 0.0491 | 22,161 | 451 | 0.0684 | 0.27 | 0.52 | Passed |
| 9 | 0.25 | 0.0491 | 15,818 | 322 | 0.0521 | 0.21 | 0.46 | Passed |
| 10 | 0.25 | 0.0491 | 14,643 | 298 | 0.0433 | 0.17 | 0.42 | Passed |
| *11 | 0.25 | 0.0491 | 10,147 | 207 | 0.0337 | 0.13 | 0.37 | Failed |
| *12 | 0.25 | 0.0491 | 9,692 | 197 | 0.0418 | 0.17 | 0.41 | Failed |
| *13 | 0.25 | 0.0491 | 7,623 | 155 | 0.0287 | 0.11 | 0.34 | Failed |
| *14 | 0.25 | 0.0491 | 9,361 | 191 | 0.0412 | 0.16 | 0.41 | Failed |
| *15 | 0.25 | 0.0491 | 9,579 | 195 | 0.0327 | 0.13 | 0.36 | Failed |
| *16 | 0.25 | 0.0491 | 7,289 | 148 | 0.0208 | 0.08 | 0.29 | Failed |
| 17 | 0.25 | 0.0491 | 15,145 | 309 | 0.0483 | 0.19 | 0.44 | Passed |
| 18 | 0.25 | 0.0491 | 10,966 | 223 | 0.0441 | 0.18 | 0.42 | Passed |
| 19 | 0.25 | 0.0491 | 13,713 | 279 | 0.0413 | 0.17 | 0.41 | Passed |
| *20 | 0.25 | 0.0491 | 8,940 | 182 | 0.0468 | 0.19 | 0.43 | Failed |
| *21 | 0.25 | 0.0491 | 9,914 | 202 | 0.0251 | 0.10 | 0.32 | Failed |
| *22 | 0.25 | 0.0491 | 11,132 | 227 | 0.0321 | 0.13 | 0.36 | Failed |
| *23 | 0.25 | 0.0491 | 13,918 | 284 | 0.0328 | 0.13 | 0.36 | Failed |
| *24 | 0.25 | 0.0491 | 9,537 | 194 | 0.0252 | 0.10 | 0.32 | Failed |
| *25 | 0.25 | 0.0491 | 7,678 | 156 | 0.0208 | 0.08 | 0.29 | Failed |
| *26 | 0.25 | 0.0491 | 25,019 | 510 | 0.0937 | 0.37 | 0.61 | Passed |

(Note)
The samples marked with * in the table are comparative examples.

As shown in Table 2, the soldering balls of Samples 1 to 3, 8 to 10, and 17 to 19 whose maximum stress before the occurrence of cracking was 2.0×10² N/mm² or more and the square root of strain ε was 0.40 or more did not have a problem with bonding reliability. On the other hand, the soldering balls of Samples 4 to 7, 11 to 16, and 20 to 25 that did not satisfy at least one of the requirement of the maximum stress and the requirement of the square root of strain ε had a problem with bonding reliability. It is to be noted that the conventional Au-based solder alloy prepared as Sample 26 had a good test result and was therefore confirmed to have high reliability, but its material cost was high due to high Au content.

### Example 2

Soldering balls were formed and their properties were evaluated in the same manner as in Example 1 described above except that the solder alloy ingots of Examples 27 and 28 were used and the diameter of the nozzle tip was adjusted so that the soldering balls of Example 27 and the soldering balls of Example 28 formed by atomization had a diameter of 0.20 mm and a diameter of 0.30 mm, respectively. The results are shown in the following Table 3.

TABLE 3

| | Ball | Cross-sectional | Fracture | Fracture | Amount of | | | |
|---|---|---|---|---|---|---|---|---|
| | diameter | area | strength | stress | displacement | Strain ε | √ε | |
| Samples | [mm] | [mm²] | [mN] | [N/mm²] | [mm] | [—] | [—] | Evaluation |
| 27 | 0.2 | 0.0314 | 14,633 | 466 | 0.0655 | 0.33 | 0.57 | Passed |
| 28 | 0.3 | 0.0707 | 32,294 | 457 | 0.0802 | 0.27 | 0.52 | Passed |

As shown in the above Table 3, even when the ball diameter of the soldering ball was changed, the soldering ball had no problem with bonding reliability as long as both the requirement of the maximum stress and the requirement of the square root of strain ε were satisfied.

### REFERENCE SIGNS LIST

1 Silicon wafer
2 Soldering ball
3 Indenter

1: An Au-based soldering ball used for sealing or bonding a ceramic electronic component, wherein when the soldering ball is crushed in one direction, a maximum stress before cracking occurs is 2.0×10² N/mm² or more, and a square root of strain before the cracking occurs is 0.40 or more.

2: The Au-based soldering ball according to claim 1, comprising 21.1% by mass or more but 43.0% by mass or less of Sn, 0.1% by mass or more but 15% by mass or less of Ag, and a balance being Au except for inevitable impurities.

3: The Au-based soldering ball according to claim 1, comprising 9.5% by mass or more but 15% by mass or less of Ge, 2% by mass or more but 10% by mass or less of Sn, and a balance being Au except for inevitable impurities.

4: The Au-based soldering ball according to claim 1, comprising 5% by mass or more but 18% by mass or less of Ag, 7% by mass or more but 20% by mass or less of Ge, and a balance being Au except for inevitable impurities.

5: A ceramic electronic component sealed using the Au-based soldering ball according to claim 1.

6: A method for evaluating bonding reliability of an Au-based soldering ball used for sealing or bonding a ceramic electronic component, the method comprising measuring whether or not a stress at which cracking is caused by crushing an Au-based soldering ball in one direction is 2.0×10² N/mm² or more, and a square root of strain before the cracking occurs is 0.40 or more.
